(19) United States
(12) Patent Application Publication
(10) Pub. No.: US 2024/0201180 A1
(43) Pub. Date: Jun. 20, 2024

(54) PLATINUM ELECTRODE SYSTEM

(71) Applicant: DIONEX CORPORATION, Sunnyvale, CA (US)

(72) Inventors: Jun Cheng, San Jose, CA (US); Yan Liu, Palo Alto, CA (US); Arshdeep Kaur, Mountain View, CA (US)

(21) Appl. No.: 18/068,219

(22) Filed: Dec. 19, 2022

(51) Int. Cl. G01N 33/543; G01N 27/30; G01N 27/327; G01N 27/404

(52) U.S. Cl. CPC G01N 33/5438; G01N 27/301; G01N 27/3273; G01N 27/4045

(57) ABSTRACT

A detection cell for a chromatography system, the detection cell includes a cell body including a counter electrode in the form of the cell body or a wire; a working electrode block including a working electrode; a gasket separating the working electrode block from the cell body, the gasket defining a sample flow pathway extending between an inlet and an outlet of the detection cell and in fluid contact with the cell body, the counter electrode, and the working electrode; and a reference electrode system in fluidic contact with the outlet and including a platinum auxiliary electrode operably connected to a positive pole of a power supply and a platinum reference electrode operably connected to a negative pole of the power supply.

### BACKGROUND

The present invention relates to, in general, flow through detection cells for chromatography detection and more particularly to solid state reference electrodes and methods for their use in such cells.

### SUMMARY

Liquid chromatographic analysis of compounds (such as carbohydrates, amino acids and related compounds) has an important place among the tools utilized in biotechnology industry, biochemical research and in clinical laboratories.

The use of liquid chromatographic columns in combination with pulsed electrochemical detection in three-electrode detection cells under alkaline conditions makes possible separations of unique selectivity and direct detection without derivatization of separated analytes with unsurpassed sensitivity.

Until now, the prevailing approach to amperometric detection in highly alkaline mobile phases uses a gold working electrode, a platinum or a titanium counter electrode, and a reference electrode of the liquid type such as a silver-silver chloride electrode, mercury-mercurous chloride electrode, mercury-mercurous sulphate electrode or thallium amalgam-thallous chloride electrode.

However, silver-silver chloride reference electrodes can undergo a change, usually a positive shift, of reference potential during their exposure to alkaline eluents used in chromatographic carbohydrate and amino acid analysis. This leads to excessive potentials being applied to the working electrodes resulting in a gradually decreasing response and/or in narrowing of range of linearity of calibration plots. In extreme cases, working electrodes can be passivated with a loss of detection sensitivity.

Other types of reference electrodes such as, for example, mercury-mercurous-chloride electrodes (calomel electrodes), mercury-mercurous sulphate electrodes, and thallium amalgam-thallous chloride electrodes (Thalamid® electrodes) can be affected by alkaline eluents in a similar fashion and affect the functioning of the working electrodes in the same way.

Also, with the increasing importance of capillary chromatography and of hyphenated detection techniques, there is a need to miniaturize the detection cells. More importantly, it becomes necessary to reduce the total cell dead volume of the electrochemical flow-through cell installed in the upstream of other detection cell. Otherwise, the too large dead volume of the electrochemical cell causes the peak efficiency significantly loss in the downstream detection cell.

However, miniaturization of cells containing the conventional common reference electrode, such as silver/silver chloride or similar reference electrode is generally very difficult due to the space requirements and surface roughness of liquid junctions and because of general bulkiness of the reference electrode body. In addition, this type of reference electrode shows other problems, such as ions leaking from the filling electrolyte solution, relative short lifetime and large total cell dead volume if used with this type of reference electrode in the miniaturized electrochemical detection cell.

On the other hand, solid state type reference electrodes can be more easily miniaturized for use with the capillary electrochemical detection cell. The total cell dead volume can be dramatically reduced to enable the cell used in front of other detection cells. Furthermore, it offers some other advantages compared to the silver/silver chloride reference electrode, for example longer lifetime, less maintenance, more robust, and ease of use.

More importantly, solid state type reference electrodes do not leak any ions like potassium and chloride in the silver/silver chloride kind of reference electrode. Therefore, multiple ED cells can be used in series and combine ED with different detection techniques, such as ED-MS for developing new applications.

The use of the solid state palladium-hydrogen reference electrodes has been described (see for example, U.S. Pat. No. 8,342,007). A constant DC power source is connected to palladium and platinum for splitting water. Hydrogen gas is generated on the palladium cathode while oxygen gas is generated on the platinum anode.

However, palladium has the ability to adsorb molecular hydrogen and volume expansion after absorbing hydrogen (Gileadi et al. Interfacial Electrochemistry: An Experimental Approach, Addison-Wesley, 1975, pp. 247-249) and is converted into various forms of palladium hydride.

It is known that three phases in palladium when hydrogen is adsorbed:
1. Alpha-phase at H:Pd atomic ratio x<0.03 (PdHx).
2. Alpha/beta (x: 0.03-0.59).
3. Beta-phase (x: >0.59).

As shown in FIG. 1, the potential plot of the palladium hydride electrode is a plateau in the phase of alpha/beta phase only. Thus, palladium hydride provides the best stable potential as a reference electrode in the alpha/beta phase (Goffe et al., “Internally Charged Palladium Hydride Reference Electrode-Part 1: The Effect of Charging Current Density on Long-Term Stability”, Med. & Biol. Eng. & Comput., 1978, 16, 670-676).

Therefore, solid-state palladium hydride reference electrodes have some intrinsic drawbacks:
1. Require too much time, two or more hours to be initialized before ready for use.
2. May shorten lifetime if used under extremely high concentration of hydroxide, i.e., 1 M NaOH.
3. Increase dimension after absorbing large volumetric quantities of hydrogen, subsequently forming palladium hydride.

Therefore, it would be desirable to provide a solid-state reference electrode that can overcome at least some of the above-mentioned issues.

The listing or discussion of an apparently prior-published document in this specification should not necessarily be taken as an acknowledgement that the document is part of the state of the art or is common general knowledge.

The present invention therefore provides a flow through detection cell for a chromatography system including a cell body having an inlet, an outlet, and a counter electrode, a working electrode, a sample flow passageway extending between the inlet and the outlet and in fluid contact with the counter and working electrodes, and a platinum hydrogen (Pt/H₂) reference electrode system.

The detection cell may be a three-electrode detection system.

The cell body may be formed of a conductive or a nonconductive material. The cell body may be formed of a corrosion resistant metal or a conductive polymer. The cell body may be formed of a material selected from the group consisting of titanium, corrosion resistant alloy, stainless steel, carbon-loaded polyetherether ketone (PEEK), polythiophene, polyindole, and polynaphthalene.

The reference electrode system is a Pt/H₂ reference electrode system. The reference electrode system includes a platinum (Pt) reference electrode and a platinum (Pt) auxiliary electrode, both reference and auxiliary electrodes being in fluid contact with the fluid sample flow passageway. The reference electrode may be connected, directly or indirectly, to a negative pole of a power supply. The auxiliary electrode may be connected, directly or indirectly, to a positive pole of a power supply.

The detection cell may further include a gasket disposed between the counter and working electrodes and having a cut out forming a thin-layer channel between the counter and working electrodes, wherein the channel may be fluidly connected to the inlet and the outlet of the cell body thereby forming a portion of the fluid sample passageway.

The cell body may include an inlet and an electrode cavity fluidly connected to the inlet and the outlet thereby forming a portion of the fluid sample flow passageway. At least one of the Pt reference electrode and the Pt auxiliary electrode may be a wire. For example, the platinum (Pt) reference electrode may include a wire extending into a reference electrode bore of the cell body insulated with a non-conductor, such as polymeric tubing.

Another aspect of the present invention is directed to a chromatography system including any of the above-described detection cells. The chromatography system may include a plurality of detection cells, wherein the detection cells may be arranged in series.

The methods and apparatuses of the present invention have other features and advantages which will be apparent from or are set forth in more detail in the accompanying drawings, which are incorporated herein, and the following Detailed Description of the Invention, which together serve to explain certain principles of the present invention.

The invention will now be described by reference to the following, non-limiting, figures and examples.

### BRIEF DESCRIPTION OF THE DRAWINGS

FIG. 1—Potential plot of the palladium hydride electrode

FIG. 2—Sideview of Thin-Layer Detection Cell with an Installed Solid-State True Pt/H₂ Reference Electrode

FIG. 3—Sideview of ED Cell with Pt/H₂ Reference Electrode in Working Electrode Block

FIG. 4—Sideview of Cell with Pt/H₂ Reference Electrode in Counter Electrode Block

FIG. 5—A schematic view of an exemplary detection cell for a chromatography system in accordance with various aspects of the present invention

FIG. 6—Overlaid Chromatograms Obtained with New Pt/H2 and Ag/AgCl REs in Current ED Cells: Mix of Six Monosaccharides

FIGS. 7A and 7B—Overlaid 40 Consecutive Chromatograms Obtained with Ag/AgCl (FIG. 7A) and Pt/H₂ (FIG. 7B) Reference Electrodes

FIGS. 8A and 8B—Response Stability Plots of 40 Consecutive Injections Obtained with Ag/AgCl (FIG. 8A) and Pt/H2 (FIG. 8B) Reference Electrodes

FIG. 9—Overlaid Chromatograms Obtained with New Pt/H₂ reference electrode in flow-through ED Cells for analysis of fluorodeoxyglucose (FDG), fluorodeoxymannose (FDM) and chlorodeoxyglucose (CDG): 0.5 ppm (solid line); 5 ppm (dotted line) and 50 ppm (dashed line)

FIG. 10—A typical chromatogram obtained with new Pt/H₂ reference electrode in flow-through ED Cells for analysis of streptomycin: 1. system suitability peak (thermal decomposition peak of streptomycin); 2. Streptomycin

FIG. 11—A typical chromatogram obtained with new Pt/H₂ reference electrode in flow-through ED Cells for analysis of mix of mono- and di-saccharides: 1. glucose; 2. fructose; and 3. Sucrose

FIG. 12—A typical chromatogram obtained with new Pt/H₂ reference electrode in flow-through ED cells for analysis of mix of seven alcohols: 1. arginine; 2. lysine; 3. alanine; 4. threonine; 5. glycine; 6. valine; 7. serine; 8. proline; 9. isoleucine; 10. leucine; 11. methionine; 12. histidine; 13. phenylalanine; 14. glutamate; 15. aspartate; 16. cystine; 17. tyrosine; *: system peak.

FIG. 13—A typical chromatogram obtained with new Pt/H₂ reference electrode in flow-through ED cells for analysis of mix of seven alcohols: 1. sorbitol; 2. glycerol; 3. ethylene glycol; 4. Methanol; 5. Ethanol; 6. 1-propanol; and 7. 1-butanol (50 ppm except for 1-butanol 100 pm); *: exclusion volume.

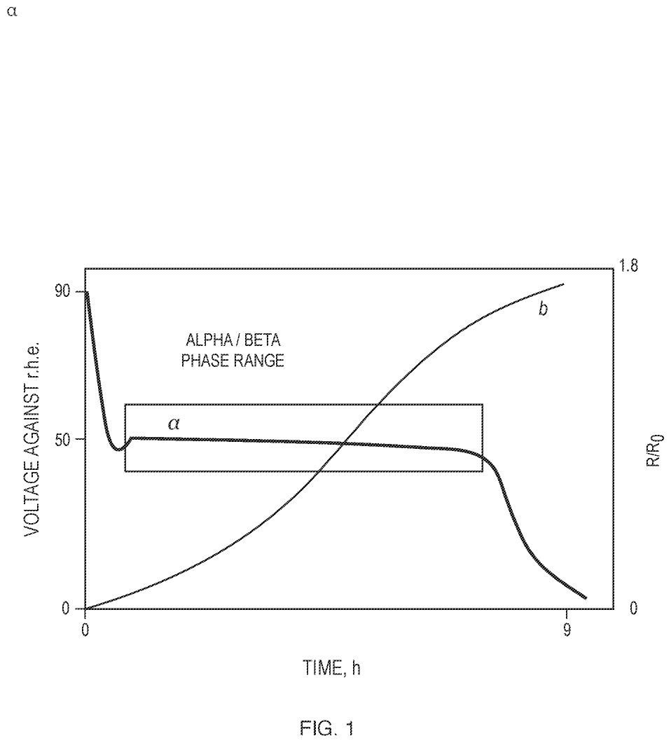

### DETAILED DESCRIPTION OF THE INVENTION

Reference will now be made in detail to various embodiments of the present invention(s), examples of which are illustrated in the accompanying drawings and described below. While the invention(s) will be described in conjunction with exemplary embodiments, it will be understood that present description is not intended to limit the invention(s) to those exemplary embodiments. On the contrary, the invention(s) is/are intended to cover not only the exemplary embodiments, but also various alternatives, modifications, equivalents and other embodiments, which may be included within the invention as defined by the appended claims.

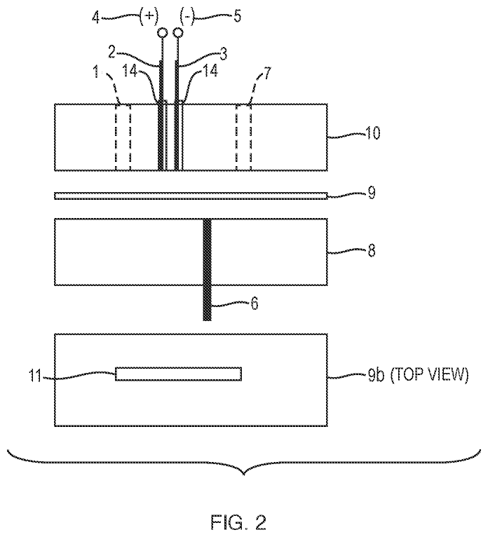

FIG. 2 illustrates an exemplary aspect of the present invention where the Pt/H₂ reference electrode is part of a three-electrode thin-layer detection cell.

The detection cell typically includes three electrodes. However, the detection cell may have two or more electrodes in accordance with various embodiments, configurations, and design considerations. The older two electrode design uses the same two electrodes (working and reference) for adjustment of voltage and for current measurement whereas in three-electrode cells only the voltage is adjusted between the reference and working electrodes. The current measurements are taken between the working electrode and the counter electrode.

The counter electrodes are called auxiliary electrodes by some. However, in the context of this text the term auxiliary electrode is reserved for the second, anodic electrode of the two-electrode system of the Pt/H₂ electrode. There are reports describing chromatographic detection cells with more than three electrodes, for example, containing multiple working electrodes referenced to the same reference electrode (LUNTE et al, “Difference Mode Detection with Thin-Layer Dual-Electrode Liquid Chromatography/Electrochemistry,” Anal. Chem., 1985, vol. 57, pp. 1541-1546). The Pt/H₂ electrode which is described here can be used with all of the above types of low dead volume chromatographic detection cells.

The detection cell of the invention has eliminated the need for a large reference electrode compartment which was required when utilising silver/silver chloride electrodes and, as shown in FIG. 2, the relatively bulky silver/silver chloride reference electrode is replaced by a Pt wire (3) connected to a negative pole (5) of a secondary power source. A positively charged electrode made of preferably Pt wire (2) is required to complete the reference electrode system (Pt/Pt charged with the secondary DC power source). The two Pt wires are insulated from each other and from the counter electrode block (10) via polymeric sleeve. The new solid-state reference electrode locates in the same thin-layer flow path as the working electrode (6) and counter electrode (10). The flow path (11) is defined by a cutout of a thin-film gasket (9) and (9b).

While it may be preferred that the Pt reference electrode (3) and/or auxiliary Pt electrode (2) are in the form of Pt wire, the Pt reference electrode (3) and/or auxiliary Pt electrode (2) may come in the shape of foil, or tube.

Typically, the detection cell will also comprise a yoke-knob assembly (not shown), for assembly of the detection cell.

Suitable materials for cell body (10) serving as the counter electrode include, but are not limited to, titanium, high quality stainless steel or sturdy high conductive polymers. On the other hand, suitable materials for the cell body not serving as the counter electrode include, but are not limited to, polytetrafluoroethylene (PTFE), polyetheretherketone (PEEK), polychlorotrifluoroethylene (Kel-F), polycarbonate. The cell body is preferably machined or otherwise formed to include an inlet (7) and an outlet (1) which are fluidly connected with thin-layer channel (11) to form a fluid sample flow line. Preferably the inlet, channel and outlet are configured so as to minimize dead volume within the detection cell. In the illustrated configuration the thin layer path of the cell is formed by the gasket's channel, however, one will appreciate that the thin-layer path of the cell can be formed by a microscopic groove machined in the body of the conductive counter electrode.

For the purposes of the present invention, thin-layer channels are those with a volume in the range from about 1 pL to about 100 μL, such as from about 1 pL to 1 μL. The following ranges of flow path dimensions either within a gasket or as a finely machined microscopic groove can be used to form the low volume, thin layer flow path. Overall dimensions of the flow path may include a width of from about 0.01 mm to about 6 mm, such as from about 0.1 to about 3 mm, a length of from about 3 mm to about 24 mm, such as from about 6 to about 12 mm, and a thickness of from about 0.001 mm to about 1.0 mm, such as from about 0.0125 to about 0.5 mm. Preferable they include a width of approximately 0.5-2 mm, length of approximately 6-10 mm, and a thickness of approximately 0.0125-0.25. Most preferably, they include a width of approximately 0.5-1.5 mm, a length of approximately 6-9 mm, and a thickness of approximately 0.0125 to 0.05 mm.

Yoke-knob assembly allows for quick assembly and disassembly of detection cell in an otherwise known manner. In particular, the yoke-knob assembly includes a yoke for aligning working electrode (6) against gasket (9), and in turn, against cell body (10) to sealingly engage the working electrode, gasket and cell body against one another. One will appreciate that the yoke-knob assembly may be configured to provide a consistent sealing force, for example, an approximately 1 to 15 lb/in2 pressure against the working electrode in order to suitably and reliably maintain the sealing action of the gasket against both the working electrode and the cell body.

As explained previously, in contrast to prior detectors which include a relatively bulky silver/silver chloride reference electrode, the detection cell of the invention includes a platinum/platinum (Pt/Pt) reference electrode system.

In FIG. 2, the platinum (Pt) hydrogen reference electrode system (2, 3) extends into a reference electrode bore of conductive cell body (10, counter electrode) insulated with polymeric tubing (14). The reference electrode (3) is connected, directly or indirectly, to a negative pole (5) of a suitable power supply.

As detailed above, the reference electrode system includes an auxiliary Pt electrode (2) to complete the reference electrode system (2, 3). The auxiliary electrode is positively charged being connected, directly or indirectly, to a positive pole (4) of a suitable power supply.

In operation and use, the detection cell is used in a manner similar to that of known detectors having a conventional silver/silver chloride reference electrode. For example, the Pt reference electrode (3) and the Pt auxiliary electrode (2) are connected to a power supply with the polarities indicated in FIG. 2. Additionally, the Pt reference electrode (3) is connected as a reference electrode to the electronic circuitry of the three-electrode detection system in an otherwise conventional manner. In a preferable configuration, the auxiliary electrode (2) is positioned downstream from the working electrode (6) and the Pt reference electrode (3) while the Pt reference electrode (3) is positioned downstream from the working electrode (6).

A Pt/H₂ reference electrode is generated by applying a potential from the power supply connected to the Pt reference (3) and Pt auxiliary (2) electrodes. Preferably, the potential is less than approximately 10 V, more preferably approximately 1.25-2 V, and most preferably approximately 1.5-1.7 V. Also, the potential is substantially constant, that is, it is subject to oscillations and/or other variations in voltage less than approximately 10 mV more preferably less than approximately 0.1 mV, and most preferably less than approximately 0.001 mV.

The Pt reference electrode provides a hydrogen electrode that gives a steady reference potential with respect to an Ag/AgCl reference electrode and a Pt/H₂ reference electrode immersed in the same solution.

The actual difference of reference values can be measured with any one of various suitable methods. For example, for any new detection experiments, a correctly adjusted value of DC amperometric and pulsed electrochemical potentials can be recognized by achieving an approximately same level of baseline signal and similar peak areas as with the silver/silver chloride reference electrode. Alternatively, the reference potential of the Pt/H₂ reference electrode can be evaluated by potential measurement compared against a conventional silver/silver chloride electrode installed in a second detection cell located downstream from the first detection cell.

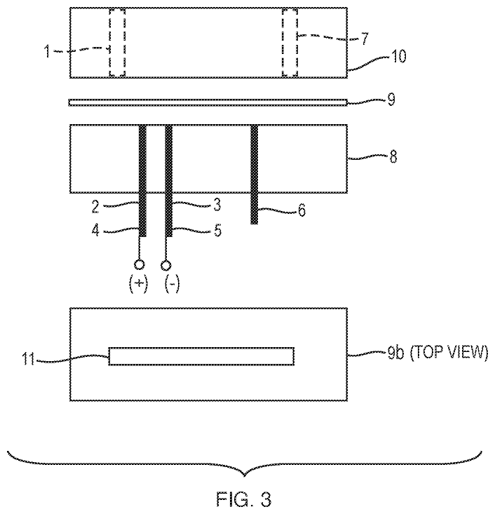

Alternatively, the Pt/H₂ reference electrode can be located within the working electrode block (8) instead, which is different from the design shown in FIG. 2 (See FIG. 3). An advantage with this design is that the Pt/H₂ reference electrode may be regenerated by polishing together with the working electrode, thereby extending the lifetime of the reference electrode.

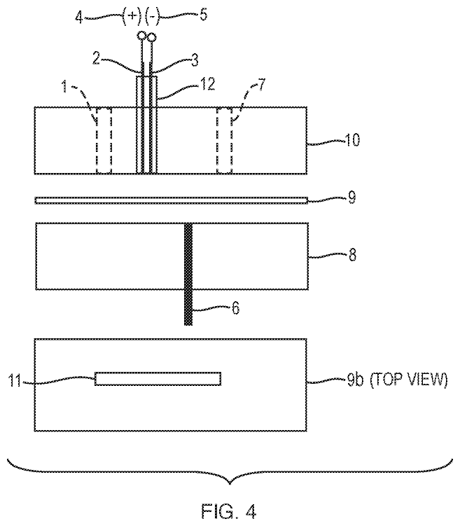

Finally, the Pt/H₂ reference electrode may be realized as the reference electrode module containing two Pt wires in very close proximity (See FIG. 4). The two Pt wires are inserted into the same polymeric tubing (12), such as dual-lumen tubing with dual holes while they are insulated from each other. Such reference electrode module can be made to fit into counter electrode and flushed with the counter electrode body in the flow-through electrochemical cell. The reference electrode is well insulated from the counter electrode (10). With such compact configuration of the Pt/H₂ reference electrode, the length of the thin-layer flow path (11) can be reduced. Consequently, the total cell dead volume of the flow-through cell is minimized even further so more likely it can prevent deterioration of the chromatographic performance on the downstream detector.

Advantageously, the present invention provides for a Pt/H₂ reference electrode which can be easily miniaturized for use in chromatographic detection cells. The present invention also allows for a Pt/H₂ reference electrode suitable for use in capillary system with extreme requirements for minimizing dead volume.

The reference electrode system of the present invention produces a stable reference potential which does not change upon exposure to eluents or mobile phases used in the chromatographic separation process. In addition, the reference electrode assembly of the present invention does not exude any ions interfering with proper functioning of other detectors connected downstream.

The configuration of the reference electrode assembly of the present invention allows for steady reference potential during long term exposure to alkaline column eluates. Advantageously, there is no accidental application of excessive detection potentials to the working electrode resulting in downward trending of detection response over time or in narrower range of linearity of calibration plots.

As the Pt/H₂ reference electrode does not include any liquid junction as a typical liquid type reference electrode, there is an absence of electrolytes exiting from the reference electrode (e.g. chloride ions exiting from the silver/silver chloride electrode). Generally, the reference electrode assembly of the present invention provides for a longer useful life as compared with conventional silver/silver chloride reference electrodes and other reference electrodes of a liquid type.

A Pt/H₂ reference electrode can be realized as a reference electrode assembly containing a Pt reference electrode (3) and a Pt auxiliary electrode (2), each in the form of a wire in close proximity with the other. Such a reference electrode system can be made to fit into existing reference electrode compartments that are designed for conventional silver/silver chloride reference electrodes.

For example, the reference electrode system a may be configured to replace the large reference electrode cylinders of existing detection cells, for example, that of the ED provided by Thermo Fisher Scientific of Waltham, Massachusetts and illustrated in ICS-6000 and other ICS-series Ion Chromatography System Operator's Manual (Dionex ICS-6000 Ion Chromatography System Operator's Manual, Document No. 22181-97002, Revision 01, February 2018) the entire contents of the ICS-6000 system manual is incorporated herein by this reference.

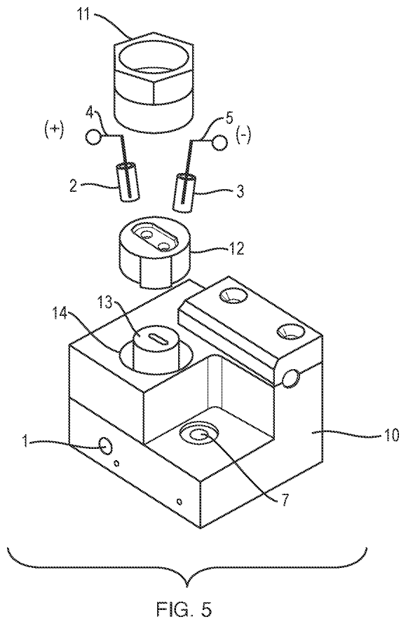

In another exemplary embodiment of the present invention illustrated in FIG. 5, the detection cell includes a cell body (10) having a threaded reference electrode cavity (14), a working electrode block (8, FIG. 4), and a gasket (9, FIG. 4) disposed between the cell body and the working electrode block. An inlet (7) is fluidly connected to the bottom of the threaded reference electrode cavity. The detection cell may be provided with a reference electrode housing that is secured within the threaded reference electrode cavity to position the ends of the electrodes in electrical contact with the fluid sample line.

The reference electrode housing may be secured within cavity (14) by a clamping nut (11), which nut allows one to releasably secure the reference electrode housing within cell body (10). One will appreciate that the electrode housing may be releasably or permanently secured within the cavity by other suitable means including, but not limited to securing gaskets, press fittings, bayonet fittings or caps, welding, and etc.

Appropriate mountings may secure the Pt reference electrode (3) and the Pt auxiliary electrode (2) within the housing. For example, 6-32 fittings and 1/16″ tubing may be utilized to sealingly secure the wire electrodes within the housing. One will appreciate that suitable means may be utilized to secure the electrodes within the reference electrode housing such as a wire sleeve. One will also appreciate that the electrodes may be formed of wire or other suitable conductors such as rod, tube, foil, net or grid shaped conductors.

A reference electrode gasket (13) may be provided which defines a thin-layer channel extending between Pt reference electrode (3) and the Pt auxiliary electrode (2). Similar to gasket (9, FIG. 4) described above, a thin-layer path of the cell by the reference electrode can be formed by a channel within reference electrode gasket or may be formed by a microscopic groove machined in reference electrode cavity (14) and/or cell body (10) of the conductive counter electrode. In either case, the channel and outlet are configured so as to minimize dead volume within the detection cell.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

For the avoidance of doubt, in this specification when we use the term “comprising” or “comprises” we mean that the detection cell or system being described must contain the listed components but may optionally contain additional components. Comprising should be considered to include the terms “consisting of’ or “consists of” where the detection cell or system being described must contain the listed component(s) only.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

The term “about” as used herein, e.g. when referring to a measurable value (such as an amount or parameter), refers to variations of ±20%, ±10%, ±5%, ±1%, ±0.5%, or, particularly, ±0.1% of the specified amount.

### EXAMPLES

The equipment used in the experiments below were obtained from Thermofisher Scientific as indicated in the Tables, and detailed information can be found in the relevant product catalogue.

Examples Obtained with Prototype Reference Electrodes on the Current ED Cell

### Example 1: Analysis of Mix of Six Monosaccharides

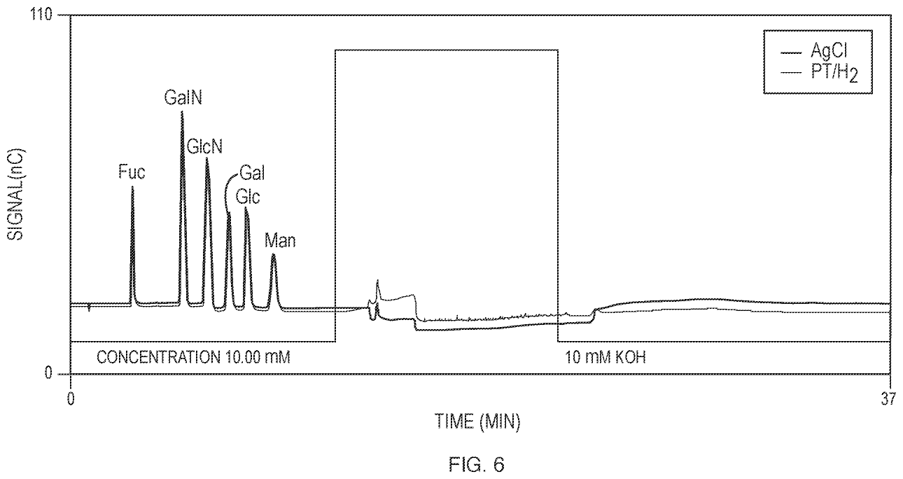

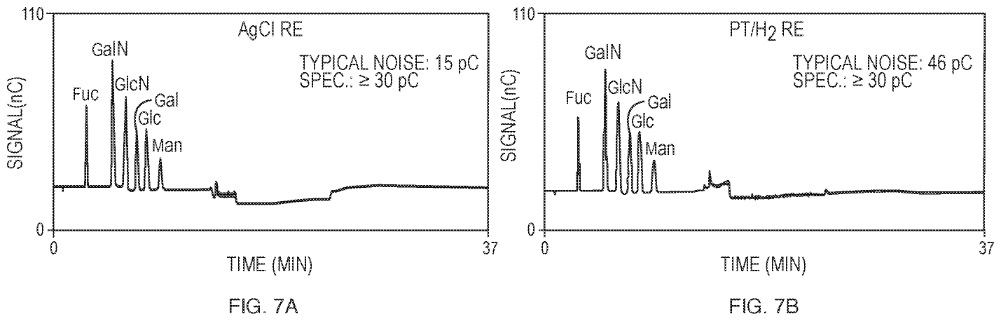

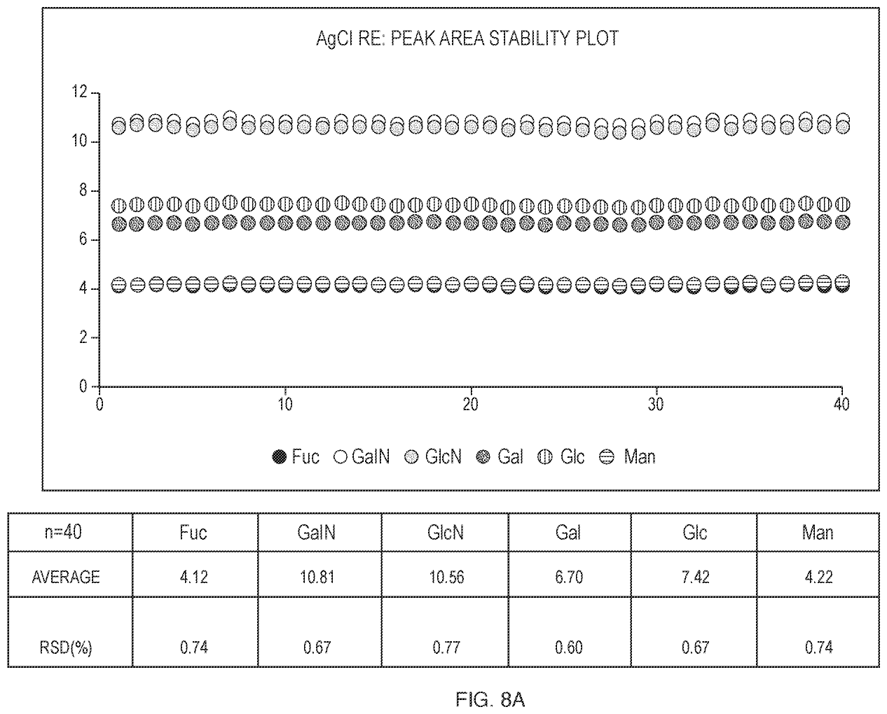

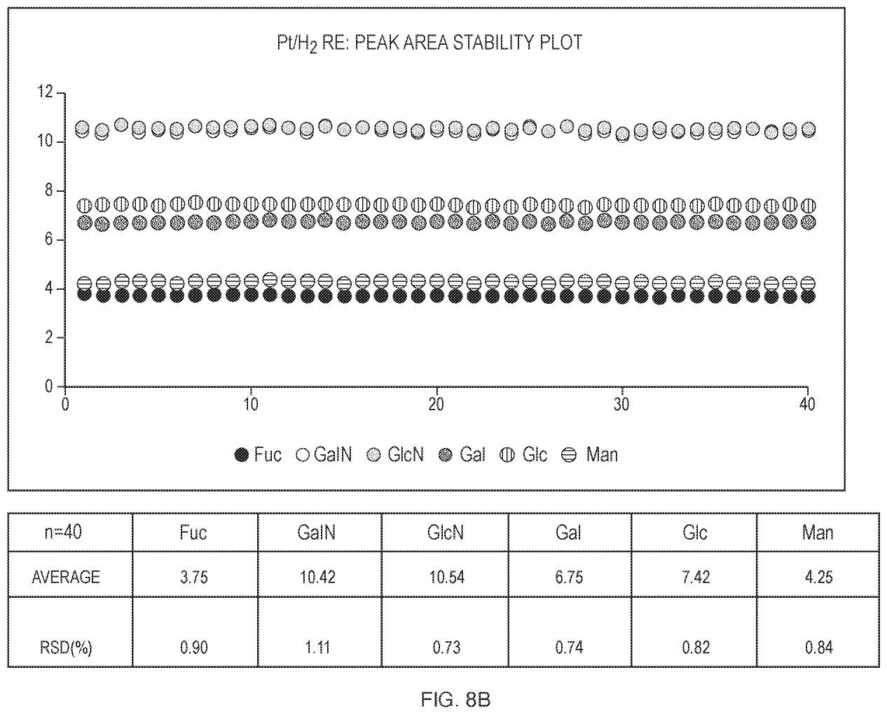

All chromatograms were generated with an ICS-5000⁺ system using the conditions of Table 1 and cell embodiment (FIG. 5). The data exemplifying the performance of new Pt/H₂ reference electrode mounted in the reference compartment of the current flow-through electrochemical cell for chromatographic detection are presented in FIGS. 6, 7A, 7B, 8A, and 8B, and Table 2.

TABLE 1
Experimental Conditions for Analysis
of Mix of Six Monosaccharides

| | |
|---|---|
| Autosampler | Dionex ™ AS-AP Autosampler |
| Eluent | 10 mM KOH with a 100 mM KOH step |
| | rinsing (KOH EGC 500 with RFIC + |
| | degassers and CR-ATC, unless |
| | otherwise specified) |
| Columns | CarboPac PA20 Analytical Column (3 × |
| | 150 mm) |
| Column Temp. | 30° C. |
| Detection Temp. | 30° C. |
| Flow Rate | 0.5 mL/min |
| Detection | Pulsed Amperometric Detection (R D. |
| | Rocklin et al., Anal. Chem. 1998, 70- |
| | 1496-1501.) |
| Gasket Thickness | 1 mil (25 μm) |
| Working electrode | Au electrode |
| Ref. Electrode | Standard Ag/AgCl or Prototype Pt/H₂ |
| Inj. Vol. | 10 μL |
| Standard | 6 Mix Monosaccharides (10 μM) |
| Run Time | 37 Minutes |

*system control and data processing: Thermo Scientific Dionex Chromeleon ® 7.2 software

TABLE 2
Performance Comparison of Ag/AgCl
and Pt/H2 Reference Electrodes

| | | | | | | | Back- | |
|---|---|---|---|---|---|---|---|---|
| | Peak Area (nC · min) | | | | | | Ground** | Noise** |
| N = 40 | Fuc | GalN | GlcN | Gal | Glc | Man | (nC) | (pC) |
| AgCl | 4.12 | 10.81 | 10.56 | 6.70 | 7.42 | 4.22 | 22 | 15 |
| Pt/H₂ | 3.75 | 10.42 | 10.54 | 6.75 | 7.42 | 4.25 | 20 | 46 |
| Ratio* | 0.91 | 0.96 | 1.00 | 1.01 | 1.00 | 1.01 | 0.91 | 3.07 |

*Ratio = Response of Prototype RE/Response of Ag/AgCl RE
**n = 3

### Example 2: Analysis of Fluorodeoxyglucose (FOG) Fluorodeoxymannose (FDM) and Chlorodeoxyglucose (CDG)

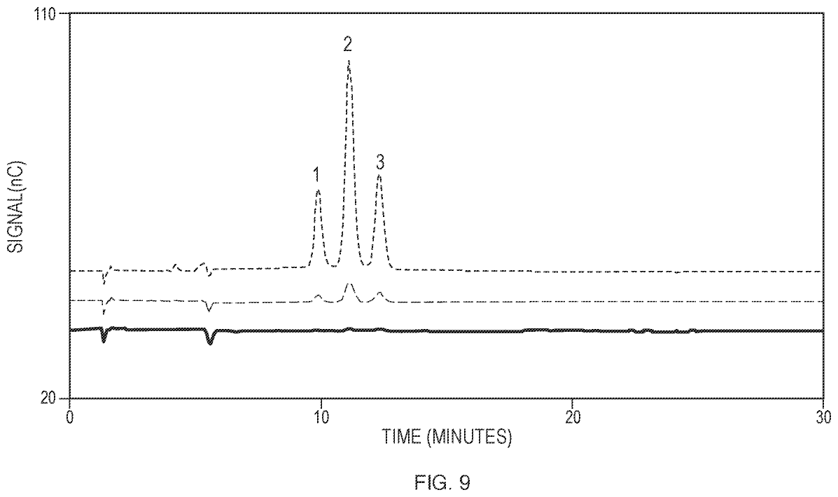

The chromatograms were generated with an ICS-6000 system using the conditions of Table 3 and cell embodiment (FIG. 5). The chromatograms exemplifying the performance of new Pt/H₂ reference electrode mounted in the reference compartment of the current flow-through electrochemical cell for chromatographic detection are presented in FIG. 9.

TABLE 3
Experimental Conditions for Analysis of Fluorodeoxyglucose (FDG),
Fluorodeoxymannose (FDM) and Chlorodeoxyglucose (CDG)

| | |
|---|---|
| Autosampler | Dionex ™ AS-AP Autosampler |
| Eluent | 100 mM KOH (KOH EGC 500 with RFIC + |
| | degassers and CR-ATC, unless |
| | otherwise specified) |
| Columns | CarboPac PA10 (Analytical 4 × 250 mm; |
| | Guard 4 × 50 mm) |
| Column Temp. | 30° C. |
| Detection Temp. | 30° C. |
| Flow Rate | 1.0 mL/min |
| Detection | Pulsed Amperometric Detection (R D. |
| | Rocklin et al., Anal. Chem. 1998, 70, |
| | 1496-1501.) |
| Gasket Thickness | 1 mil (25 μm) |
| Working electrode | Au electrode |
| Ref. Electrode | Prototype Pt/H₂ |
| Inj. Vol. | 10 μL |
| Standard | FDG, FDM and CDG |

*system control and data processing: Thermo Scientific Dionex Chromeleon ® 7.2 software

### Example 3: Analysis of Streptomycin

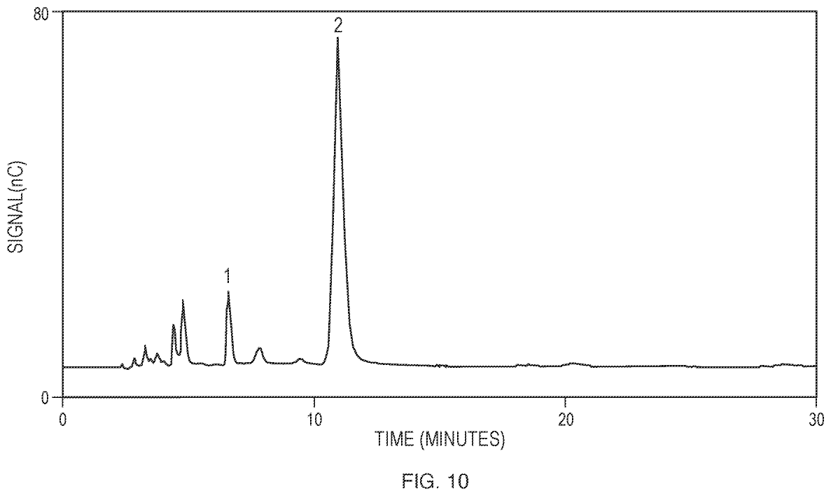

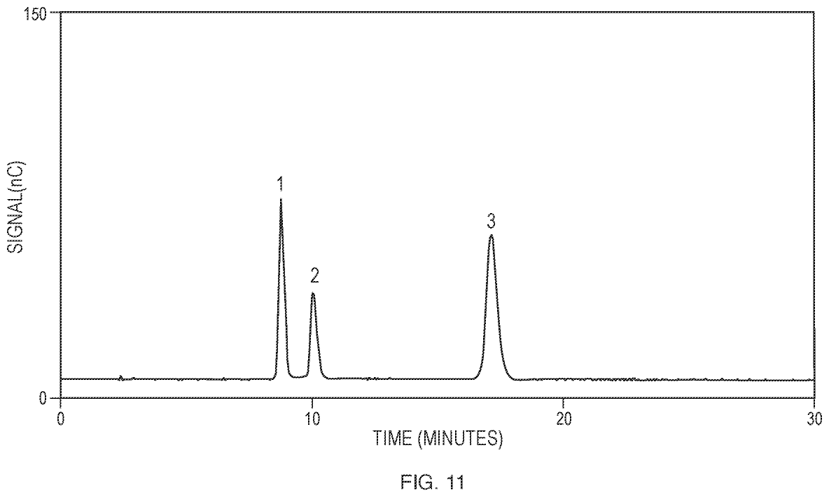

The chromatogram was generated with an ICS-6000 system using the conditions of Table 4 and cell embodiment (FIG. 2). The chromatogram exemplifying the performance of new Pt/H₂ reference electrode mounted in the reference compartment of the current flow-through electrochemical cell for chromatographic detection is presented in FIG. 10.

TABLE 4
Experimental Conditions for Analysis of Streptomycin

| | |
|---|---|
| Autosampler | Dionex ™ AS-AP Autosampler |
| Eluent | 70 mM NaOH (A: water, B: 250 mM |
| | NaOH, 72% A/28% B) |
| Columns | CarboPac PA1 (Anal. 4 × 250 mm) |
| Column Temp. | 30° C. |
| Detection Temp. | 30° C. |
| Flow Rate | 0.5 mL/min |
| Detection | Pulsed Amperometric Detection (R D. |
| | Rocklin et al., Anal. Chem. 1998, 70, |
| | 1496-1501.) |
| Gasket Thickness | 2 mil (50 μm) |
| Working electrode | Disposable Au of PTFE |
| Ref. Electrode | Prototype Pt/H₂ |
| Inj. Vol. | 10 μL |
| Standard | Steptomycin |

*system control and data processing: Thermo Scientific Dionex Chromeleon ® 7.2 software

### Example 4: Analysis of Mix of Mono- and Di-Saccharides

The chromatogram was generated with an ICS-6000 system using the conditions of Table 5 and cell embodiment (FIG. 2). The chromatogram exemplifying the performance of new Pt/H₂ reference electrode mounted in the reference compartment of the current flow-through electrochemical cell for chromatographic detection is presented in FIG. 11.

TABLE 5
Experimental Conditions for Analysis
of Mono- and Di-mosaccharides

| | |
|---|---|
| Autosampler | Dionex ™ AS-AP Autosampler |
| Eluent | 100 mM NaOH (A: water, B: 250 mM |
| | NaOH, 60% A/40% B) |
| Columns | CarboPac PA1 (Anal. 4 × 250 mm) |
| Column Temp. | 30° C. |
| Detection Temp. | 30° C. |
| Flow Rate | 0.5 mL/min |
| Detection | Pulsed Amperometric Detection (R D. |
| | Rocklin et al., Anal. Chem. 1998, 70, |
| | 1496-1501.) |
| Gasket Thickness | 2 mil (50 μm) |
| Working electrode | Disposable Au of PTFE |
| Ref. Electrode | Prototype Pt/H₂ |
| Inj. Vol. | 10 μL |
| Standard | Glu, Frc and Suc |

*system control and data processing: Thermo Scientific Dionex Chromeleon ® 7.2 software

### Example 5: Analysis of 17 Amino Acids

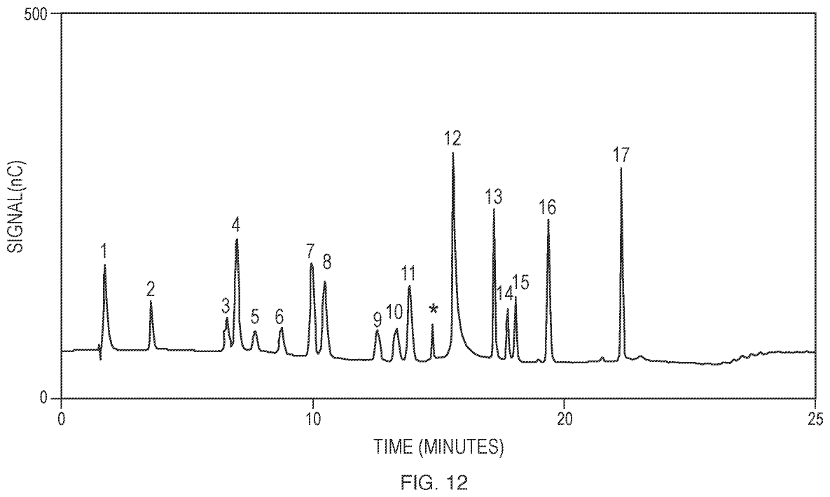

The chromatogram was generated with an ICS-6000 system using the conditions of Table 6 and cell embodiment (FIG. 5). The chromatogram exemplifying the performance of new Pt/H₂ reference electrode mounted in the reference compartment of the current flow-through electrochemical cell for chromatographic detection is presented in FIG. 12.

TABLE 6
Experimental Conditions for Analysis of 17 amino acids

| | |
|---|---|
| Autosampler | Dionex ™ AS-AP Autosampler |
| Eluent | H₂O (A)/0.25 NaOH (B)/1M NaAc (C) |
| | (gradient) |
| Columns | AminoPac PA10 (Anal. 2 × 250 mm; |
| | Guard 2 × 50 mm) |
| Column Temp. | 30° C. |
| Detection Temp. | 30° C. |
| Flow Rate | 0.25 mL/min |
| Detection | Integrated Pulsed Amperometric |
| | Detection (J. Cheng et al., Anal. Chem. |
| | 2003, 75, 572-579) |
| Gasket Thickness | 2 mil (50 μm) |
| Working electrode | Disposable Pt |
| Ref. Electrode | Prototype Pt/H₂ |
| Inj. Vol. | 25 μL |
| Standard | 17 Amino Acids |
| Run Time | 25 min |

*system control and data processing: Thermo Scientific Dionex Chromeleon ® 7.2 software

### Example 6: Analysis of Mix of Seven Alcohols

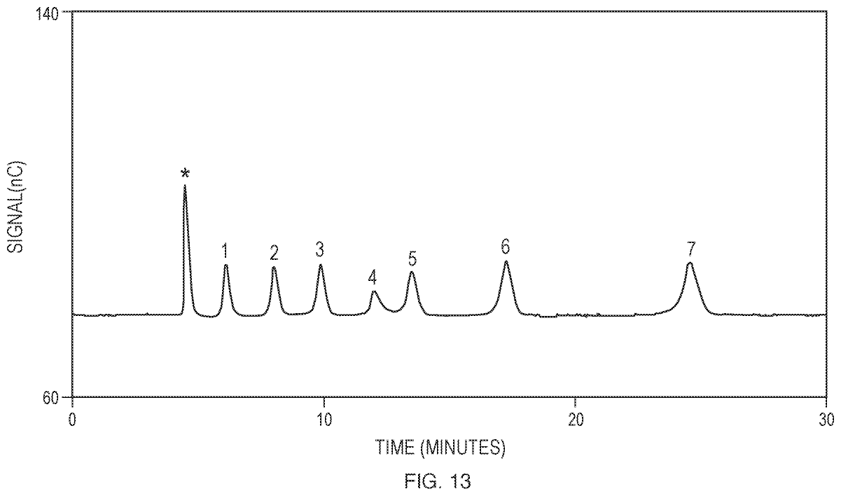

The chromatogram was generated with an ICS-6000 system using the conditions of Table 7 and cell embodiment (FIG. 5). The chromatogram exemplifying the performance of new Pt/H₂ reference electrode mounted in the reference compartment of the current flow-through electrochemical cell for chromatographic detection is presented in FIG. 13.

TABLE 7
Experimental Conditions for Analysis of Mix of Seven Alcohols

| | |
|---|---|
| Autosampler | Dionex ™ AS-AP Autosampler |
| Eluent | 100 mM MSA |
| Columns | IonPac (4 × 250 mm) |
| Column Temp. | 30° C. |
| Detection Temp. | 30° C. |
| Flow Rate | 0.20 mL/min |
| Detection | Pulsed Amperometric Detection (J. |
| | Cheng et al., J. Electroanal. Chem. 2007, |
| | 608, 117-124.) |
| Gasket Thickness | 2 mil (50 μm) |
| Working electrode | Disposable Pt |
| Ref. Electrode | Prototype Pt/H₂ |
| Inj. Vol. | 20 μL |
| Standard | Sorbitol, glycerol, ethylene glycol, |
| | methanol, ethanol, 1-propanol and 1- |
| | butanol. |

*system control and data processing: Thermo Scientific Dionex Chromeleon ® 7.2 software

1. A detection cell for a chromatography system, the detection cell comprising:
a cell body including a counter electrode in the form of the cell body or a wire;
a working electrode block including a working electrode;
a gasket separating the working electrode block from the cell body, the gasket defining a sample flow pathway extending between an inlet and an outlet of the detection cell and in fluid contact with the cell body, the counter electrode, and the working electrode; and
a reference electrode system in fluidic contact with the outlet and including a platinum auxiliary electrode operably connected to a positive pole of a power supply and a platinum reference electrode operably connected to a negative pole of the power supply.

2. A detection cell according to claim 1, wherein the detection cell is a three-electrode detection system.

3. A detection cell according to claim 1, wherein the cell body is formed of a conductive material or nonconductive material.

4. A detection cell according to claim 1, wherein the cell body is formed of a corrosion resistant metal or a conductive polymer and a nonconductive polymer.

5. A detection cell according to claim 4, wherein the cell body is formed of a conductive material selected from the group consisting of titanium, corrosion resistant alloy, stainless steel, carbon-loaded polyetherether ketone (PEEK), polythiophene, polyindole, and polynaphtalene while a nonconductive material selected from the group consisting of polytetrafluoroethylene (PTFE), polyetheretherketone (PEEK), polychlorotrifluoroethylene (Kel-F), polycarbonate.

6. A detection cell according to claim 1, wherein the reference electrode system includes a solid state reference electrode.

7. A detection cell according to claim 1, wherein the platinum reference electrode and the Pt auxiliary electrode are in electrical contact with the fluid sample passageway.

8. A detection cell according to claim 7, wherein the reference electrode includes a wire extending into a reference electrode bore of the cell body.

9. A detection cell according to claim 1, wherein the cell body includes an electrode cavity fluidly connected to the inlet and the outlet thereby forming a portion of the fluid sample passageway.

10. A detection cell according to claim 9, wherein at least one of the Pt/H₂ reference electrode and the platinum auxiliary electrode is a wire.

11. A chromatography system comprising the detection cell according to claim 1.

12. A chromatography system comprising the detection cell according to claim 9.

13. A chromatography system comprising a plurality of detection cells according to claim 1, wherein the detection cells are arranged in series.

14. A detection cell for a chromatography system, the detection cell comprising:
a cell body;
a working electrode block including a working electrode;
a sample channel extending between an inlet and an outlet of the detection cell and in fluid contact with the cell body and the working electrode;
a counter electrode in fluid contact with the sample channel; and
a reference electrode system in fluidic contact with the outlet and including an auxiliary electrode operably connected to a positive pole of a power supply and a Pt/H₂ reference electrode operably connected to a negative pole of the power supply.

15. A detection cell according to claim 14, wherein the cell body is formed of a conductive material.

16. A detection cell according to claim 14, wherein the Pt/H₂ reference electrode and the auxiliary electrode are in electrical contact with the sample channel, and in which the auxiliary electrode includes a platinum electrode.

17. A detection cell according to claim 14, wherein the Pt/H₂ reference electrode includes a wire extending into a reference electrode bore of the cell body.

18. A detection cell according to claim 14, wherein the reference electrode system operates at a potential less than approximately 10V subject to variations in voltage of less than approximately 10 mV.

19. A detection cell according to claim 14, wherein the channel has a volume of approximately 1 pL to 100 μL.
